(19) United States
(12) Patent Application Publication
(10) Pub. No.: US 2004/0111111 A1
(43) Pub. Date: Jun. 10, 2004

(54) Intravascular filter membrane with shape memory

(75) Inventor: Horng-Ban Lin, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SciMed Life Systems, Inc.

(21) Appl. No.: 10/315,830

(22) Filed: Dec. 10, 2002

(51) Int. Cl. A61M 29/00

(52) U.S. Cl. 606/200

(57) ABSTRACT

An intravascular device can capture embolic debris. An intravascular filter can employ a shape memory filter membrane. In particular, an intravascular filter membrane can be designed for deployment in a vascular system. The filter membrane can be made of a shape memory polymer, and the membrane filter can be moveable between a collapsed insertion configuration and an expanded deployment configuration. The shape memory polymer remembers the expanded deployment configuration.

### TECHNICAL FIELD

The invention relates generally to intravascular devices and more particularly to emboli-capturing devices. In particular, the invention relates to emboli-capturing devices having shape memory characteristics.

### BACKGROUND

Heart and vascular disease are major problems throughout the world. Conditions such as atherosclerosis result in blood vessels becoming blocked or narrowed. Occluded, stenotic, or narrowed blood vessels can be treated with a number of relatively non-invasive medical procedures including percutaneous transluminal angioplasty (PTA), percutaneous transluminal coronary angioplasty (PTCA), and atherectomy.

During angioplasty and atherectomy procedures, embolic debris can be separated from the wall of the blood vessel. If this debris enters the circulatory system, it could block other vascular regions including the neural and pulmonary vasculature. During angioplasty procedures, stenotic debris may also break loose due to manipulation of the blood vessel. Because of this debris, a number of devices, termed embolic protection devices, have been developed to filter out this debris.

Typical embolic protection devices employ a membrane that is supported and configured by a metal frame. The metal frame is responsible for deploying the membrane. The metal frame, and thus the membrane, can have a collapsed configuration for insertion and an expanded configuration upon deployment. The collapsed configuration has a minimal profile, for ease of insertion. The expanded configuration has a larger profile, intended to bring an outer edge of the membrane into contact with the vessel lumen in which it is employed. Body temperature can cause the metal frame to move from its collapsed configuration to a remembered deployment configuration if the metal frame is constructed from a shape memory alloy.

It is possible that after being in a compressed configuration, the membrane may not return completely to the original or intended deployment configuration even, with a metallic shape memory frame. This reduces the efficiency of the filter. Thus, a need remains for an improved embolic protection device.

### SUMMARY

The present invention describes an intravascular device that captures embolic debris. In broad terms, the invention describes an intravascular filter that employs a shape memory polymer filter membrane.

Accordingly, an embodiment of the present invention is found in an intravascular filter membrane that is designed for deployment in a vascular system. The filter membrane is made of a shape memory polymer, and the membrane filter is moveable between a collapsed insertion configuration and an expanded deployment configuration. The shape memory polymer remembers the expanded deployment configuration.

An embodiment of the present invention is found in an intravascular filter membrane that has an insertion configuration and a deployment configuration. The intravascular filter membrane is formed from a shape memory polymer that has a glass transition temperature of less than about 37 degrees C., The polymer membrane is shaped into the deployment configuration at a temperature at or above about 37 degrees C. which locks the deployment configuration into memory. The polymer membrane is cooled to ambient temperature, and is subsequently deformed into the insertion configuration.

An embodiment of the present invention is found in an intravascular filter assembly that includes a frame and a filter membrane that is disposed on the frame. The filter membrane has an insertion configuration and a deployment configuration and is formed of a shape memory polymer that remembers the deployment configuration and changes from the insertion configuration to the deployment configuration upon heating to about 37 degrees C.

An embodiment of the present invention is found in a method of forming an intravascular filter membrane that has an insertion configuration and a deployment configuration. A polymer membrane formed of a shape memory polymer that has a glass transition temperature of less than about 37 degrees C. is shaped into the deployment configuration at a temperature at or above about 37 degrees C., thereby locking the deployment configuration into memory. The polymer membrane is cooled to ambient temperature and subsequently is deformed the insertion configuration.

### Brief Description of the Drawings

FIG. 1 is a perspective view of an embodiment of an intravascular filter assembly in an expanded deployment configuration;

FIG. 2 is a view of the intravascular filter assembly of FIG. 1, shown in a partially collapsed configuration;

FIG. 3 is a view of the intravascular filter assembly of FIG. 1, shown in its collapsed configuration, being inserted through a vessel via an insertion sheath;

FIG. 4 is a view of the intravascular filter assembly of FIG. 1, shown in its expanded deployment configuration and illustrating a retrieval sheath for retrieving the filter assembly;

FIG. 5 is a perspective view of an embodiment of an intravascular filter assembly, shown coupled to a guidewire in its expanded, deployed configuration;

FIG. 6 is a view of the intravascular filter assembly of FIG. 5, shown in a vessel;

FIG. 7 is a view of an embodiment of an intravascular filter assembly;

FIG. 8 is a view of an embodiment of an intravascular filter assembly, shown in an expanded deployment configuration; and

FIG. 9 is a view of the intravascular filter of FIG. 8, shown in a collapsed insertion configuration.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Shape Memory Polymers

In broad terms, shape memory polymers behave similarly to shape memory alloys such as the nickel-titanium alloys commonly referred to as Nitinol. The material is formed in its parent shape and is heated to a temperature that is at or above the glass transition temperature of the material. After the material has cooled, perhaps to ambient temperature, the material can be molded into any desired shape that is within the mechanical limitations of the material. This shape is temporarily retained until the material is once again subjected to its transition temperature. If desired, this process of low temperature deformation followed by thermally induced recovery of the parent shape can be repeated indefinitely.

One feature of shape memory polymers is that they have a large and reversible change in the modulus of elasticity of the material between the lower temperature glassy (crystalline) region and the higher temperature rubbery (elastic) region. In some embodiments, this large change in elasticity can be represented by a ratio of modulus of elasticity (below T_{g}) to modulus of elasticity (above T_{g}) of at least about 20.

In one aspect, shape memory polymers can be considered as having hard segments that are typically crystalline in nature, and soft segments that are typically amorphous in nature. However, some hard segments can be amorphous, and some soft segments can be crystalline. In this, segment refers to a block or sequence of the polymer that forms part of the shape memory polymer.

The terms hard segment and soft segment are relative in nature, and refer to differences in the transition temperatures of the segments, with a hard segment having the higher transition temperature. A shape memory polymer can have a first set of soft segments having a first transition temperature, and a second set of soft segments having a different, second transition temperature. In this case, the shape memory polymer can remember two distinct shapes that will be retrieved at different temperatures. The nature of shape memory polymers is discussed in greater detail in U.S. Pat. Nos. 6,160,084 and 6,388,043, each of which are incorporated in their entirety by reference herein.

In another aspect, the characteristics of shape memory polymers can be considered in terms of Brownian motion. In particular, molecular chains can undergo micro-Brownian motion above the glass transition temperature, once the modulus of elasticity has dropped. As noted above, shape memory polymers are considered as exhibiting a large drop in the modulus of elasticity when heating through the glass transition temperature.

In the elastic or rubbery state, the material can be easily deformed via mechanical means. As a result of the deformation, the molecular chains will orient themselves in line with the tension. Subsequently lowering the temperature below the glass transition temperature of the material freezes the micro-Brownian motion and therefore locks the material in its deformed configuration. The material will retain its deformed configuration for as long as the material remains below the glass transition temperature of the material.

When the material is heated above the glass transition temperature, however, micro-Brownian motion begins again, and the molecular chains will move to reduce or eliminate the tension caused by the initial deformation. As a result, the material will regain its remembered shape.

To function as a shape memory polymer, it is advantageous that the material either be partially crystallized or include at least some crosslinking. It has been found, however, that even when the material is partially crystallized or crosslinked, it can still be melted and processed using conventional injection or extrusion molding equipment.

Further, the polymers previously used to make filter membranes did not have a partial crystalline or crosslinked structure that is needed to possess the shape memory property. Prior art processing of these polymers using conventional techniques, such as molding, extrusion, casting, etc., do not induce shape memory property either. On the other hand, the shape memory polymers contain a partial crystalline or crosslinked structure, and the structure has been found to be maintained after being processed by conventional techniques. Thus, the shape memory polymers of the present invention possess the shape memory property after being processed.

The transition temperature of a shape memory polymer can be adjusted by varying the ratio of polymers used to create the shape memory polymer. A variety of different polymers can be made to have shape memory characteristics. Examples of suitable polymers include polynorborene (available commercially from Nippon Zeon Company), trans-polyisoprene (available from Kuraray Company), styrene-butadiene (available from Ashahi Company) and polyurethane (available from Mitsubishi Heavy Industries).

Additional materials that can be used include poly L-D lactic copolymer, oligo caprylactone copolymer and poly cyclo-octine. These polymers can be used separately or in conjunction with other shape memory polymers. In embodiments where more than one shape memory polymer is used, it is preferred that the polymers are compatible and that the glass transitions are similar.

Shape memory polymers typically have three-dimensional networks as interpolymer chain interactions are important in retaining stable shapes. Examples of interpolymer chain interactions include chain entanglement, chemical cross-linking, and crystal, aggregate or glassy state formation. Entanglement and crosslinking are permanent changes and are used for constructing the original shape, while the other chain interactions are thermally reversible and thus are used to maintain the temporary (deformed) shapes.

For example, polynorborene relies on entanglement for memorizing an original shape, while trans-polyisoprene and polyethylene rely on crosslinking for this purpose. Polyurethane and styrene-butadiene copolymer rely on the formation of micro crystals in remembering an original shape. With respect to maintaining a deformed (temporary) shape, polynorborene and polyurethane employ the formation of a glass state. Trans-polyisoprene, styrene-butadiene copolymer and polyethylene each rely on the formation of micro-crystals.

Use of Shape Memory Polymer

An intravascular filter membrane that is designed for deployment in a vascular system can be made from a shape memory polymer, resulting in a membrane that is moveable between a collapsed insertion configuration and an expanded deployment configuration. The collapsed insertion configuration can represent a temporary deformed shape, and expanded deployment configuration can represent a permanent remembered shape.

A filter membrane can have a collapsed profile while in its collapsed insertion configuration and a deployed profile in its expanded deployment configuration, and the collapsed profile is smaller than the deployed profile. The collapsed insertion configuration can be adapted for insertion via a catheter delivery system or delivery sheath. The expanded deployment configuration can be adapted to at least substantially occlude a portion of the vascular system in which the intravascular filter membrane is deployed.

A filter membrane can have an open proximal end and a closed distal end, and the open proximal end can include a thickened annular ring that is adapted to provide an adequate level of hoop strength when the filter membrane is in the deployed configuration.

A shape memory polymer can be a polymer that remembers a memorized shape when it reaches a temperature that is greater than ambient temperature but is less than or about equal to human body temperature. The shape memory polymer can be a polymer that exhibits a large reversible change in its modulus of elasticity at its glass transition temperature. The polymer can have a glass transition temperature of less than about 37 degrees C.

An intravascular filter membrane can be formed by providing a polymer membrane made from a shape memory polymer that has a glass transition temperature of less than about 37 degrees C. and shaping the polymer membrane into a deployment configuration at a temperature at or above about 37 degrees C. Once the polymer membrane has cooled to ambient temperature, the polymer membrane can be deformed into an insertion configuration.

The shape memory polymer can have a glass transition temperature that is in the range of about 30 to about 35 degrees C. A polymer membrane can be shaped into a deployed configuration at a temperature that is in the range of about 45 to about 60 degrees C. The insertion configuration of a polymer membrane can be obtained via mechanical deformation of the polymer membrane.

A polymer membrane can regain a remembered deployment configuration upon subsequent heating to a temperature of about 37 degrees C. The remembered deployment configuration can be substantially identical to the locked in deployment configuration.

In a particular embodiment, the insertion configuration can represent a first memorized shape, and the deployment configuration can represent a second memorized shape. Each of the insertion configuration and the deployment configuration can independently be manifested as a result of thermal changes.

An intravascular filter assembly can include a frame and a shape memory polymer filter membrane. The filter membrane has an insertion configuration and a deployment configuration and remembers the deployment configuration (thereby changing from the insertion configuration to the deployment configuration) upon heating to about 37 degrees C.

The frame can be made from a shape memory material, such as a shape memory alloy or a shape memory polymer. The frame can be made from Nitinol. The frame can be made from a shape memory polymer and can be integrally formed with the filter membrane.

Intravascular Filter Assemblies

Intravascular filter assemblies can be formed having a variety of different configurations. Some configurations can be considered as having an umbrella-style frame. Other configurations can be considered as having a hoop-style frame. Yet other configurations can be considered as having a helical string-style frame. Intravascular filters can be constructed without a separate frame. The frame can be integral with the membrane, or the membrane alone can provide the necessary hoop strength. Each of these general configuration types is discussed hereinafter and can be constructed using the shape memory polymers described herein.

Umbrella Configuration

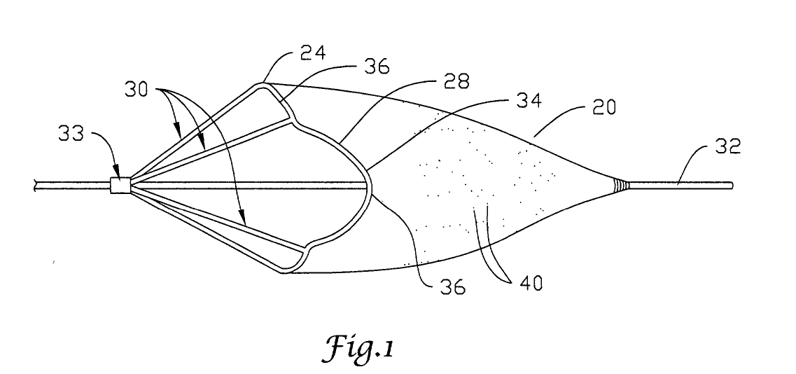

As noted, one general class of distal protection devices or emboli filters includes those having umbrella-style frames. FIGS. 1 through 4 illustrate an intravascular filter assembly that includes such a frame.

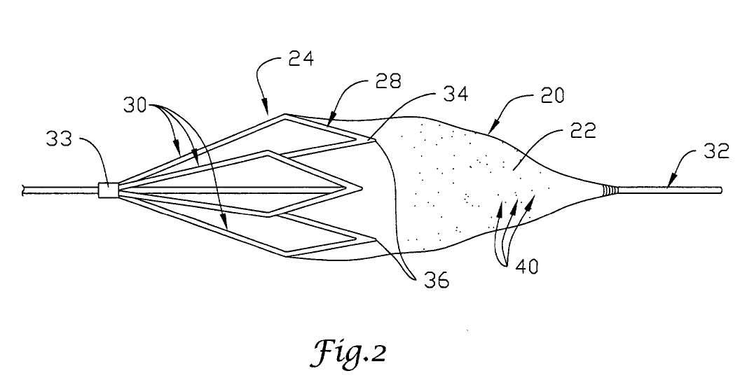

As illustrated comparatively in FIGS. 1-2, the intravascular filter assembly 20 operatively moves between a closed collapsed profile, adapted for insertion into a body lumen as illustrated in FIG. 2, and an open radially-expanded deployed profile for collecting debris in a body lumen as illustrated in FIG. 1.

The intravascular filter assembly 20 includes a filter 22 and a collapsible proximally-tapered frame 24. The frame 24 supports the filter 22 and can be operably coupled to an elongated guidewire 32 or other support device. The frame 24 includes a mouth 28 and a plurality of longitudinally-extending ribs 30. In an expanded profile, the mouth 28 is opened and the ribs extend radially outwardly to support the mouth 28. A collar 33 can movably couple the proximal ends of the ribs 30 to the guidewire 32.

The filter 22 can be cone-shaped, having a proximal and a distal end. The distal end can be a narrow, “V”-shaped end and can be fixedly secured or formed to the guidewire 32. The proximal end can have a relatively wide opening and can be coupled to the mouth 28 of the frame 24. The filter 22 can be formed of a shape memory polymer. The filter 22 can be formed of a shape memory polymer in which a collapsed, insertion profile represents a temporary deformation and in which an expanded, deployed profile represents a remembered shape that can be regained once the filter 22 reaches body temperature.

In particular, the filter 22 can be formed of a porous shape memory material having a plurality of small openings 40. The holes or openings 40 can be sized to allow blood flow therethrough, but restrict flow of debris or emboli floating in the body lumen or cavity. In the embodiment shown, the guidewire 32 extends through the mouth 28 of the intravascular filter assembly 20 and along the entire length of the device and is fixed to the distal end of the filter 22.

The mouth 28 can be formed of a pleated ring 34 having an expanded dimension to support the filter 22 in the opened deployed profile and a collapsed dimension to support the filter in the closed collapsed profile. In the opened expanded profile, the ring 34 includes a plurality of folds 36 that are spaced so that the diameter of the pleated ring 34 forms a mouth of sufficient diameter so that an opening to the filter 22 conforms to a desired body lumen. The pleated ring 34 is collapsed by closing the folds 36 so that adjacent folds 36 are positioned in close proximity. In such a position, the mouth 28 assumes a relatively small dimension to collapse the filter 22 for insertion and retrieval.

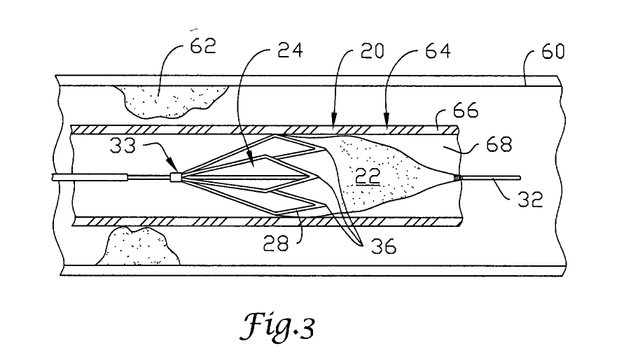

As shown in FIG. 3, the intravascular filter assembly 20 is first collapsed and inserted in the collapsed profile into a delivery sheath 64. The sheath 64 can be formed of a tubular member 66 including an inner lumen 68 extending therethrough. The profile of sheath 64 is relatively small to facilitate insertion and placement of the intravascular filter assembly 20, which is placed in lumen 68 for insertion. Once the intravascular filter assembly 20 is inside the delivery sheath 64, the sheath 64 can be inserted through the vasculature of a patient and has its distal end positioned distal of the stenosis or blocked region 62.

To deploy the intravascular filter assembly 20 after it is suitably located, the sheath 64 is withdrawn, thus permitting the folds 36 resiliently separate to open the mouth 28 and the filter 22 for operation. The mouth 28 can be sized so that when the folds 36 separate, the mouth 28 conforms to the dimensions of the vascular lumen 60. The mouth 28 supports the filter 22 relative to the circumference of the vascular lumen 60 so that blood flows through the filter and debris and particles floating in the blood are trapped by the filter.

The frame 28 can be formed of a Nitinol alloy or other elastic material so that the frame “springs” back to an expanded profile after the confining force imparted via the sheath 64 is released. The frame 28 can be formed of a shape memory polymer that has a glass transition temperature at or below normal body temperature. Thus, once the intravascular filter assembly 20 has been inserted and the frame 28 has been exposed to body temperature, the frame 28 can revert to a remembered expanded profile.

The relatively elastic material provides sufficient resilient force for a tight interaction between the mouth 28 and the lumen 60 to assure that blood flows through the filter 22 to capture floating debris and particles.

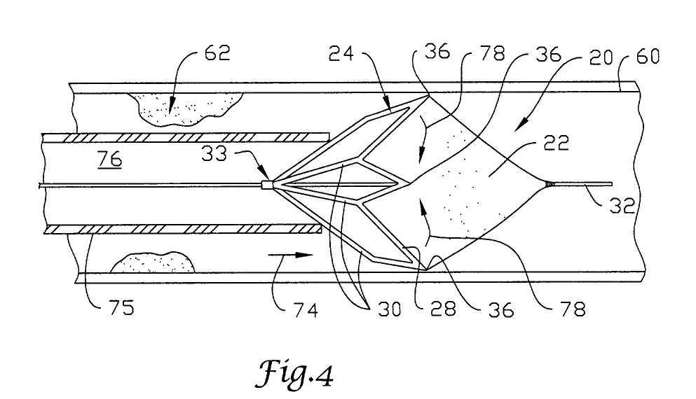

After deployment, the sheath 64 can be withdrawn and various treatment devices, such as an angioplasty dilatation catheter, stent delivery catheter or other atherectomy or thrombectomy devices, can be employed. Treatment devices can be inserted over guidewire 32 for placement relative to the treatment site. After treatment is complete, the intravascular filter assembly 20 is removed as illustrated in FIG. 4.

As shown in FIG. 4, a retrieval sheath 72 is inserted as illustrated via arrow 74 for removal of the intravascular filter assembly 20. The retrieval sheath 72 is formed of a tubular member 75 having a central lumen 76 and a distal opening sized to capture the intravascular filter assembly 20. The retrieval sheath 72 can be inserted to align the distal opening of the sheath 72 with the proximal end of frame 24. Thereafter, the sheath 72 can be advanced or, alternatively, as illustrated, the guidewire 32 can be retracted to collapse ribs 30, thereby collapsing mouth the 28 and the filter 22 as illustrated by arrows 78. As the ribs 30 collapse inwardly, the frame 24 folds at the folds 36 until the mouth 28 resides within or closely proximate the distal end of the sheath 72, thereby trapping emboli therein.

Hoop Configuration

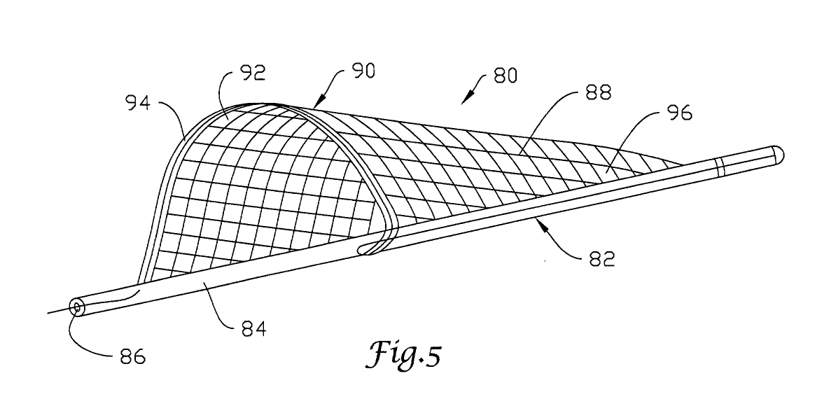

As noted, one general class of distal protection devices or emboli filters includes those having hoop-style frames. FIGS. 5 through 8 illustrate an intravascular filter assembly that includes such a frame.

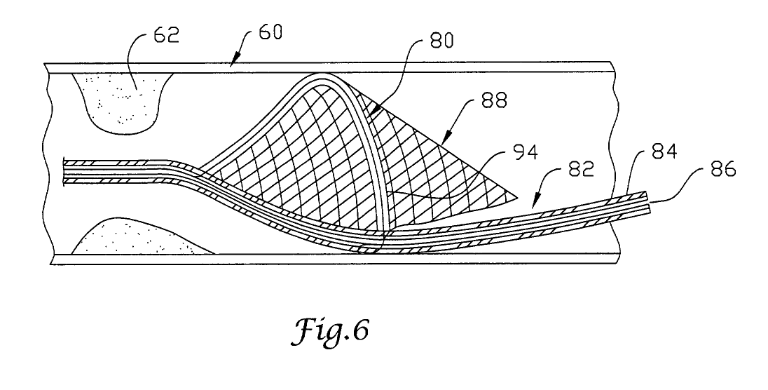

FIGS. 5 and 6 illustrate an intravascular filter assembly 80. The intravascular filter assembly 80 can be coupled to a guidewire 82 to operate between a radially-expanded deployed profile and a collapsed profile for insertion and retrieval. The guidewire 82 is formed of a tubular member 84 including a central lumen 86 therethrough. The guidewire 82 can be formed of a hypo tube or other material. The intravascular filter assembly 80 includes a filter 88 and a frame 90.

The frame 90 can be formed of an elongate wire 92 and a polymer sleeve 94. The frame 90 is coupled to the guidewire 82 and is supported thereby. The filter 88 is coupled to the frame 90 and is supported thereby at its proximal end by the frame 90.

The filter 88 can be formed of a shape memory polymer having holes or openings 96 therein to allow blood to flow therethrough while restricting flow of emboli, debris and clotting material. The filter 88 can be formed of a shape memory polymer in which a collapsed, insertion profile represents a temporary deformation and in which an expanded, deployed profile represents a remembered shape that can be regained once the filter 88 reaches body temperature.

The filter 88 can be cone-shaped, with a “V”-shaped tip and a large opening to funnel debris for collection. The filter 88 and the sleeve 94 can be integrally or separately formed, and secured via known attachment methods.

As can be seen in FIG. 6, the intravascular filter assembly 80 can be inserted in a low-profile dimension at a deployment site, preferably distal of a stenosis 62. As the mouth of the intravascular filter assembly 80 expands to conform to the vascular dimension, guidewire 82 pushes against a lumen wall to provide a tight fit between the filter 88 and the vascular wall 60.

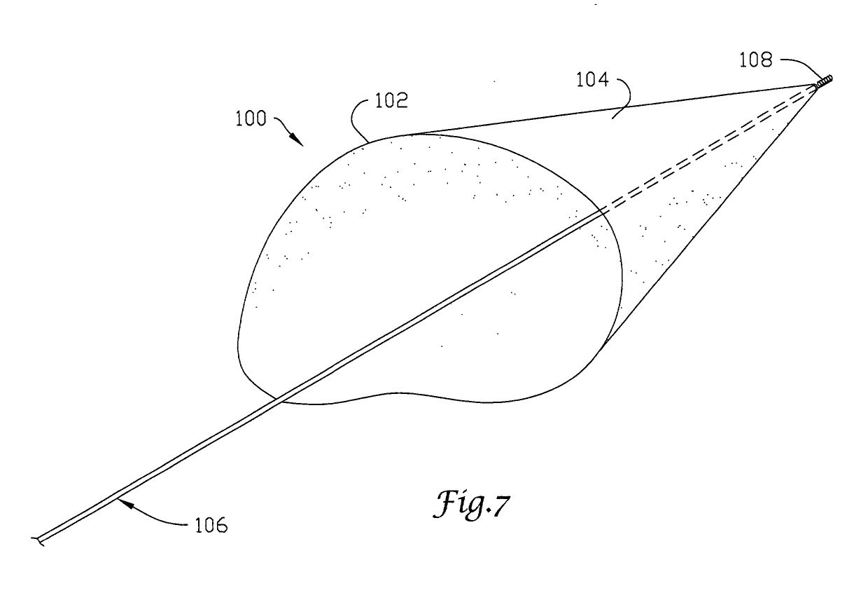

FIG. 7 illustrates an intravascular filter assembly 100. The intravascular filter assembly 100 includes a hoop-shaped frame 102, a filter membrane 104, and a wire 106. The hoop-shaped frame 102 can be a self-expanding frame formed of a wire which includes a shape memory alloy. The hoop-shaped frame 102 can be formed of a nitinol wire. The hoop-shaped frame 102 can be formed of a shape memory polymer that has a collapsed insertion profile and an expanded, deployment profile. The hoop-shaped frame 102 can be a separate element that is formed and subsequently attached to the filter membrane 104. The hoop-shaped frame 102 can be an integrally-formed portion of the filter 104 that represents an annular thickening or ring-shaped thickening that adds hoop strength to the intravascular filter assembly 100.

The filter portion 104 can be formed of a polyurethane material having holes therein such that blood flow can pass through filter 104, but emboli (of a desired size) cannot pass through filter 104 but are retained therein. The filter material 104 can be attached to the hoop-shaped frame 102 with a suitable, commercially available adhesive. The filter 104 can have a proximal portion that is folded over the hoop-shaped frame 102 and is attached either with adhesive, by stitching, or by another suitable connection mechanism, in order to secure it about the hoop-shaped frame 102. The distal end of the filter 104 can be attached about the outer periphery of wire 106, proximate a coil tip 108 on the wire 106.

The filter 104 can be formed of a polyurethane material with the holes laser drilled therein. The holes are preferably approximately 100 micrometers in diameter. The filter 104 can also be a microporous membrane, a wire or polymer braid or mesh, or any other suitable configuration. The filter 104 can be formed of a shape memory polymer in which a collapsed, insertion profile represents a temporary deformation and in which an expanded, deployed profile represents a remembered shape that can be regained once the filter 104 reaches body temperature.

If it is desired to make the wire 106, the hoop 102, or the filter 104 radiopaque, other materials can be used. For example, radiopaque loaded powder can be used to form a polyurethane sheath which is fitted over the wire 106 or the hoop 102, or which is implemented in the filter 104.

In operation, the hoop 102 (and thus the filter 104) can be collapsed to a radially contracted position that more closely approximates the outer diameter of the wire 106. Once retracted to a more low profile position, the wire 106 can be manipulated to position the hoop 102 and the filter 104 distal of a restriction to be treated. Then, the restraining force which is used to restrain the hoop 102 in the predeployment, low profile position is removed, and the superelastic properties of the nitinol hoop 102 (or the shape memory properties of another shape memory alloy) are utilized in allowing the hoop 102 to assume its shape memory position. This causes the hoop 102 to define a substantially lumen filling mouth to the filter 104 which is positioned distal of the restriction to be treated.

A suitable dilatation device is then advanced over the wire 106 and is used to treat the vascular restriction. Emboli which are carried by blood flow distal of the restriction are captured by the filter 104. After the dilatation procedure, the filter 104, along with the emboli retained therein, are retrieved from the vasculature. Various retrieval procedures and devices are described later in the specification.

By allowing the hoop-shaped frame 102 to be unattached to the wire 106, and only connected to the wire 106 through the filter 104, the wire 106 is allowed to substantially float within the hoop 102. This configuration provides some advantages.

For instance, the hoop 102 can better follow the vasculature without kinking or prolapsing (i.e., without collapsing upon itself).

Helical Configuration

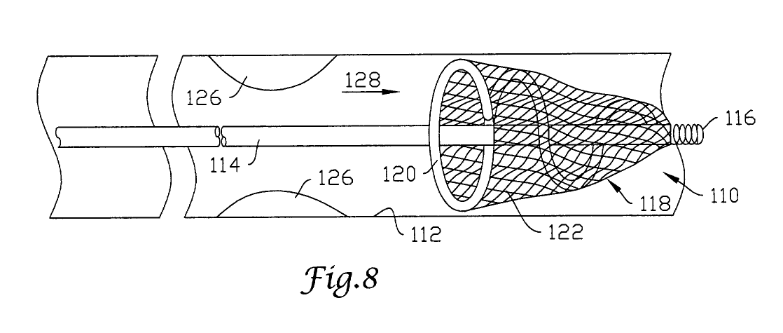

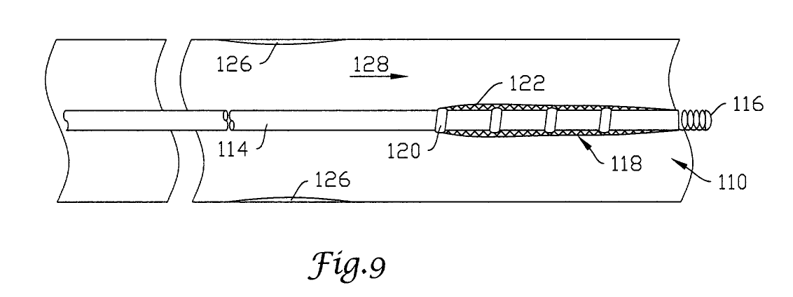

As noted, one general class of distal protection devices or emboli filters includes those having helical-style frames or activating members. FIGS. 8 and 9 illustrate an intravascular filter assembly that includes such a frame.

FIG. 8 illustrates an intravascular filter assembly 110 in a deployed position within the lumen of a blood vessel 112. The intravascular filter assembly 110 can include a hollow guidewire 114 having a coil tip 116, and a filter membrane 118, which can include an expandable member 120 and mesh 122. When deployed, the expandable member 120 expands to the position shown in FIG. 8 such that the filter membrane 118 has an outer periphery that approximates the inner periphery of the lumen 112.

The mesh 122 can be formed of woven or braided fibers or wires, or a microporous membrane, or other suitable filtering or netting-type material. The mesh 122 can be formed of a shape memory polymer in which a collapsed, insertion profile represents a temporary deformation and in which an expanded, deployed profile represents a remembered shape that can be regained once the mesh 122 reaches body temperature.

The mesh 122 can be a microporous membrane having holes therein with a diameter of approximately 100 micrometers. The mesh 122 can be formed of a single generally cone-shaped piece which is secured to the outer or inner periphery of the expandable member 120. Alternatively, the mesh 122 can be formed as a spiral strip which is secured about the outer or inner periphery of the expandable member 120 filling the gaps between the loops of the expandable member 120. The mesh 122 can be formed of a number of discrete pieces that are assembled onto the expandable member 120.

Upon expansion, the expandable member 120 expands radially outwardly from the outer surface of the guidewire 114 and carries the mesh 122 into the deployed position shown in FIG. 8. In this way, the filter membrane 118 can be deployed distally of the stenosis 126 so that the stenosis 126 can be severed and fragmented, and so fragments from the stenosis 126 can be carried by blood flow (indicated by arrow 128) into the basket or chamber formed by the deployed filter membrane 118. The filter membrane 118 can then be collapsed and removed from the vessel 112 with the fragments of the stenosis 126 contained therein.

FIG. 9 illustrates the intravascular filter assembly 110 with the filter membrane 118 in the collapsed position. FIG. 9 illustrates that the mesh 122 is easily collapsible with the expandable member 120. The expandable member 120 can be formed of a material having some shape memory characteristics.

In particular, the expandable member 120 can be formed from a shape memory material such as a shape memory alloy or a shape memory polymer as described herein. The expandable member 120 illustrated in FIG. 9 can be formed from a shape memory polymer in which the collapsed position represents a temporary deformation in the polymer, while the expanded position illustrated in FIG. 8 can represent a memorized shape that can be obtained once the intravascular filter assembly 110 has been deployed within a vessel and has reached body temperature.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What we claim is:

1. An intravascular filter membrane for deployment in a vascular system, the filter membrane comprising a shape memory polymer, the membrane moveable between a collapsed insertion configuration and an expanded deployment configuration, wherein the polymer remembers the expanded deployment configuration.

2. The intravascular filter membrane of claim 1, wherein the membrane has a collapsed profile while in its collapsed insertion configuration and a deployed profile in its expanded deployment configuration, and the collapsed profile is smaller than the deployed profile.

3. The intravascular filter membrane of claim 1, wherein the membrane in its collapsed insertion configuration is adapted for insertion via a catheter delivery system.

4. The intravascular filter membrane of claim 1, wherein the membrane in its expanded deployment configuration is adapted to at least substantially occlude a portion of the vascular system in which the intravascular filter membrane is deployed.

5. The intravascular filter membrane of claim 1, wherein the filter membrane has an open proximal end and a closed distal end.

6. The intravascular filter membrane of claim 5, wherein the open proximal end comprises a thickened annular ring adapted to provide an adequate level of hoop strength when the filter membrane is in the deployed configuration.

7. The intravascular filter membrane of claim 1, wherein the shape memory polymer comprises a polymer that remembers a memorized shape when it reaches a temperature that is greater than ambient temperature but is less than or about equal to human body temperature.

8. The intravascular filter membrane of claim 1, wherein the shape memory polymer comprises a polymer that exhibits a large reversible change in its modulus of elasticity at its glass transition temperature.

9. The intravascular filter membrane of claim 1, wherein the shape memory polymer comprise a polymer having a glass transition temperature of less than about 37 degrees C.

10. The intravascular filter membrane of claim 1, wherein the shape memory polymer comprises one of polyurethane, polynorborene, trans-polyisoprene, styrene-butadiene copolymer, or dimethylacrylate-butyl acrylate copolymer.

11. The intravascular filter membrane of claim 10, wherein the shape memory polymer comprises polyurethane.

12. An intravascular filter membrane having an insertion configuration and a deployment configuration, formed by the process of:
providing a polymer membrane comprising a shape memory polymer having a glass transition temperature of less than about 37 degrees C.;
shaping the polymer membrane into the deployment configuration at a temperature at or above about 37 degrees C., thereby locking the deployment configuration into memory;
cooling the polymer membrane to ambient temperature; and
deforming the polymer membrane into the insertion configuration.

13. The intravascular filter membrane of claim 12, wherein the shape memory polymer has a glass transition temperature that is in the range of about 30 to about 35 degrees C.

14. The intravascular filter membrane of claim 12, wherein the polymer membrane is shaped into the deployed configuration at a temperature that is in the range of about 45 to about 60 degrees C.

15. The intravascular filter membrane of claim 12, wherein the insertion configuration of the polymer membrane is adapted for placement within a catheter deployment system.

16. The intravascular filter membrane of claim 12, wherein the insertion configuration is obtained via mechanical deformation of the polymer membrane.

17. The intravascular filter membrane of claim 12, wherein the insertion configuration represents a first memorized shape and the deployment configuration represents a second memorized shape; where the insertion configuration and the deployment configuration are independently manifested as a result of thermal changes.

18. The intravascular filter membrane of claim 12, wherein the polymer membrane regains a remembered deployment configuration upon subsequent heating to a temperature of about 37 degrees C.

19. The intravascular filter membrane of claim 18, wherein the remembered deployment configuration is substantially identical to the locked in deployment configuration.

20. An intravascular filter assembly comprising:
a frame; and
a filter membrane disposed on the frame, the filter membrane having an insertion configuration and a deployment configuration;
wherein the filter membrane is formed of a shape memory polymer that remembers the deployment configuration and changes from the insertion configuration to the deployment configuration upon heating to about 37 degrees C.

21. The intravascular filter assembly of claim 20, wherein the open end has a first diameter in the insertion configuration and a second diameter in the deployment configuration, the second diameter being greater than the first diameter.

22. The intravascular filter assembly of claim 20, wherein the frame comprises a shape memory material.

23. The intravascular filter assembly of claim 22, wherein the shape memory material comprises a shape memory alloy or a shape memory polymer.

24. The intravascular filter assembly of claim 23, wherein the frame comprises nitinol.

25. The intravascular filter assembly of claim 23, wherein the frame comprises a shape memory polymer and is integrally formed with the filter membrane.

26. A method of forming an intravascular filter membrane that has an insertion configuration and a deployment configuration, the method comprising:
providing a polymer membrane comprising a shape memory polymer having a glass transition temperature of less than about 37 degrees C.;
shaping the polymer membrane into the deployment configuration at a temperature at or above about 37 degrees C., thereby locking the deployment configuration into memory;
cooling the polymer membrane to ambient temperature; and
deforming the polymer membrane into the insertion configuration.

27. The method of claim 26, wherein the shape memory polymer has a glass transition temperature that is in the range of about 30 to about 35 degrees C.

28. The method of claim 26, wherein the polymer membrane is shaped into the deployed configuration at a temperature that is in the range of about 45 to about 60 degrees C.

29. The method of claim 26, wherein the insertion configuration of the polymer membrane is adapted for placement within a catheter deployment system.

30. The method of claim 26, wherein the insertion configuration is obtained via mechanical deformation of the polymer membrane.

31. The method of claim 26, wherein the insertion configuration represents a first memorized shape and the deployment configuration represents a second memorized shape; where the insertion configuration and the deployment configuration are independently manifested as a result of thermal changes.

32. The method of claim 26, wherein the polymer membrane regains a remembered deployment configuration upon subsequent heating to a temperature of about 37 degrees C.

33. The method of claim 26, wherein the remembered deployment configuration is substantially identical to the locked in deployment configuration.
